(19) United States
(12) Patent Application Publication
(10) Pub. No.: US 2022/0378683 A1
(43) Pub. Date: Dec. 1, 2022

(54) COSMETIC COMPOSITION COMPRISING VITAMIN C

(71) Applicant: L'OREAL, Paris (FR)

(72) Inventors: Nicholas David STEBBINS, Clark, NJ (US); David CHAN, Oradell, NJ (US)

(73) Assignee: L'OREAL, Paris (FR)

(21) Appl. No.: 17/329,742

(22) Filed: May 25, 2021

(51) Int. Cl. A61K 8/67; A61K 8/06; A61K 8/44; A61K 8/35; A61K 8/37; A61Q 19/00; A61Q 19/08; A61Q 19/02

(52) U.S. Cl. CPC A61K 8/676; A61K 8/062; A61K 8/44; A61K 8/35; A61K 8/375; A61Q 19/00; A61Q 19/08; A61Q 19/02; A61K 2800/51; A61K 2800/522

(57) ABSTRACT

A cosmetic composition including (a) from about 2 to about 20 wt. % of ascorbic acid; (b) di-t-butyl pentaerythrityl tetrahydroxycinnamate; (c) hydroxyacetophenone; and (d) a chelating agent; and wherein the cosmetic composition is an oil-in-water emulsion, and all weight percentages are based on the total weight of the cosmetic composition. Methods for treating damaged skin with cosmetic compositions are also disclosed herein.

### FIELD OF THE INVENTION

The present disclosure relates to cosmetic compositions comprising high amounts of ascorbic acid (vitamin C), products containing the cosmetic compositions, and methods for treating skin using the cosmetic compositions.

### BACKGROUND OF THE INVENTION

Skin acts as a natural barrier between internal and external environments and therefore plays an important role in vital biological functions such as protection against mechanical and chemical injury, microorganisms, and ultraviolet damage. The health and appearance of skin, however, can deteriorate due to environmental factors, genetic makeup, nutrition, and sun exposure.

Environmental pollution conditions are fast worsening and becoming more apparent in the daily life of consumers worldwide. The damage of pollution against human skin is also becoming more and more evident. Human skin is also subjected to a variety of insults by extrinsic factors such as ultraviolet (UV) radiation, environmental pollution, wind, heat, infrared radiation, low humidity, harsh surfactants, abrasives, etc. Recent studies suggest that in addition to UV radiation, other environmental factors contribute to the development of solar lentigines, particularly air pollution. Ultimately, these factors result in visible signs of skin damage including small brown patches on the skin, especially in the elderly.

Typical skin damage includes fine lines, wrinkling, hyperpigmentation, sallowness, sagging, dark under-eye circles, puffy eyes, enlarged pores, visible dead skin, i.e., flaking, scaling, dryness, and roughness. Consumers desire to slow the gaining of skin damage and reduce the effects of aging, especially in the face and around the eyes. Radiant and clear skin appears youthful and is a sign of good health and vitality. Accordingly, there is an ongoing need for new and improved formulations that improve the health and visual appearance of skin.

Vitamin C is a potent antioxidant that can be used topically in dermatology to treat and prevent changes associated with photoageing, hyperpigmentation, etc. Vitimin C is very unstable and difficult to formulate and deliver into the dermis. Research has been ongoing to develop stable compositions that deliver high amounts of ascorbic acid to the skin.

### SUMMARY OF THE DISCLOSURE

The instant disclosure relates to cosmetic compositions that are unique in comparison to conventional cosmetic compositions because they contain and deliver a high concentration of ascorbic acid. Ascorbic acid is very difficult to stabilize, and therefore pose unique formulations challenges.

The inventors discovered that certain ingredients in specific amounts enables cosmetic compositions containing high level of ascorbic acid to be stable.

While not wishing to be bound by any particular theory, the inventors believe that di-t-butyl pentaerythrityl tetrahydroxycinnamate, hydroxyacetophenone, and a chelating agent, in combination with other components of the compositions, contribute to the stability and effectiveness of the compositions. The cosmetic compositions typically include:

(a) from about 2 to about 20 wt. % of ascorbic acid;

(b) di-t-butyl pentaerythrityl tetrahydroxycinnamate;

(c) hydroxyacetophenone; and

(d) a chelating agent; and

wherein the cosmetic composition is an oil-in-water emulsion, and all weight percentages are based on the total weight of the cosmetic composition.

The cosmetic compositions may be in the form of an emulsion, for example, an oil-in-water emulsion. In some embodiments, the cosmetic composition is in the form of an oil-in-water emulsion.

In some embodiments, the di-t-butyl pentaerythrityl tetrahydroxycinnamate is present from about 0.05 to about 0.5 wt. % based on the total weight of the cosmetic composition.

In one or more embodiments, the hydroxyacetophenone is present from about 0.1 to about 3 wt. % based on the total weight of the cosmetic composition.

In some embodiments, the chelating agent is chosen from Trisodium Ethylenediamine Disuccinate, Sodium Phytate, Tetrasodium Glutamate Diacetate, and mixture thereof. In one or more embodiments, the chelating agent is Trisodium Ethylenediamine Disuccinate. In one embodiment, the chelating agent is present from about 0.01 to about 1 wt. % based on the total weight of the cosmetic composition.

In some embodiments, the cosmetic composition may include one or more fatty compounds. Non-limiting examples of suitable fatty compounds may include fatty alcohols, fatty acids, fatty esters, oils, waxes, derivatives thereof, and mixtures thereof. In various embodiments, the one or more fatty ester may be chosen from ethoxylated fatty esters, sorbitan fatty esters, esters of stearates, esters of behenates, esters of arachidates, esters of palmitates, fatty acid esters of a sugar, and mixtures thereof. In one embodiment, one or more fatty ester may be chosen from purcellin oil (cetearyl octanoate), isopropyl myristate, isopropyl palmitate, C12-C15 alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, and pentaerythritol esters, and mixtures thereof. In some embodiments, the one or more fatty compounds comprises isononyl isononanoate, caprylic/capric triglyceride, Glycine soja (soybean) oil or mixtures thereof.

In some embodiment, the oil phase may comprise from about 0.5 to about 20 wt. %, based on the total weight of the cosmetic composition.

Additionally or alternatively, the cosmetic composition may include one or more polyols. In various embodiments, the one or more polyols are chosen from caprylyl glycol, pentylene glycol, butylene glycol, hexalyne glycol, glycerin, dipropylene glycol, propylene glycol, and mixtures thereof.

In some cases, the cosmetic composition is free of tocopherol.

In accordance with another aspect of the disclosure, the cosmetic composition may include:

(a) from about 2 to about 20 wt. % of ascorbic acid;

(b) from about 0.05 to about 0.5 wt. % of di-t-butyl pentaerythrityl tetrahydroxycinnamate;

(c) from about 0.01 to about 3 wt. % of hydroxyacetophenone; and

(d) from about 0.01 to about 2 wt. % of a chelating agent chosen from Trisodium Ethylenediamine Disuccinate, Sodium Phytate, Tetrasodium Glutamate Diacetate; and

wherein the cosmetic composition is an oil-in-water emulsion, and all weight percentages are based on the total weight of the cosmetic composition.

The cosmetic compositions are useful for treating skin, for example, the skin of the face and neck of a human. Thus, the instant disclosure relates to methods of treating the skin comprising application of the cosmetic composition of the instant disclosure to the skin. The cosmetic compositions are additionally useful in methods for treating skin dryness, repairing skin damage due to photoaging, and diminishing the appearance of wrinkles, dark spots, and uneven skin texture. The aforementioned methods may be non-therapeutic.

### DETAILED DESCRIPTION OF THE DISCLOSURE

The present disclosure relates to stable cosmetic compositions that contain high amounts of ascorbic acid (vitamin C), and methods for treating skin using the cosmetic compositions. Ascorbic acid is a naturally occurring antioxidant. It is an enhancer of the biosynthesis of collagen, which is the protein that provides shape and firmness to the skin. Because ascorbic acid leads to higher production of collagen, wrinkling in skin is minimized. In addition, ascorbic acid can decrease age spots by reversing hyperpigmentation in skin cells to a certain extent by inhibiting tyrosinase activity.

The cosmetic compositions of the instant disclosure typically include:

(a) from about 2 to about 20 wt. % of ascorbic acid;

(b) di-t-butyl pentaerythrityl tetrahydroxycinnamate;

(c) hydroxyacetophenone; and

(d) a chelating agent; and

wherein the cosmetic composition is an oil-in-water emulsion, and all weight percentages are based on the total weight of the cosmetic composition.

The cosmetic compositions are generally formulated as an emulsion. Typically, the cosmetic compositions are formulated to be oil-in-water emulsions.

In one embodiment, the composition is an oil-in-water emulsion. For example, the amount of oil phase may be from about 0.5, about 1, about 1.5, about 2, about 2.5, about 3, about 3.5, about 4, about 5, about 6, about 7, about 8, about 9, about 10 to about 10, about 11, about 12, about 13, about 14, about 14.5, about 15, about 15.5, about 16, about 16.5, about 17, about 17.5, about 18, about 18.5, about 19, about 19.5, or about 20 wt. %, including ranges and sub-ranges there between, based on the total weight of the composition. Additionally or alternatively, the amount of oil phase may be 0.5, 1, 1.5, 2, 2.5, 3, 3.5, 4, 5, 6, 7, 8, 9, 10 to about 10, 11, 12, 13, 14, 14.5, 15, 15.5, 16, 16.5, 17, 17.5, 18, 18.5, 19, 19.5, or 20 wt. %, including ranges and sub-ranges there between, based on the total weight of the composition. In one embodiment, the amount of oil phase may be 10% or about 10% of the total weight of the cosmetic composition.

As discussed above, it is believed that the di-t-butyl pentaerythrityl tetrahydroxycinnamate, hydroxyacetophenone and a chelating agent advantageously facilitate the stabilization of the ascorbic acid in the emulsion. The aqueous phase of the cosmetic composition may include an amount of ascorbic acid. For instance, a majority or essentially the entirety of ascorbic acid of the cosmetic composition may reside in the aqueous phase.

Suitable components, such as those listed below, may be included or excluded from the formulations for the cosmetic compositions depending on the specific combination of other components, the form of the cosmetic compositions, and/or the use of the formulation (e.g., a lotion, a serum, gel, cream, etc.).

Ascorbic Acid

The cosmetic compositions include ascorbic acid.

The ascorbic acid is pure L-ascorbic acid (vitamin C), and is typically present in the cosmetic composition in an amount of from about 2 wt. % to about 20 wt. %, based on the total weight of the composition. For example, the cosmetic compositions may include an amount of ascorbic acid ranging from about 2.0 wt. % to about 20 wt. %, about 2.5 wt. % to about 25 wt. %, about 3.5 wt. % to about 20 wt. %, about 4.5 wt. % to about 20 wt. %, about 5 wt. % to about 15 wt. %, about 6 wt. % to about 14 wt. %, about 7 wt. % to about 13 wt. %, or about 8 wt. % to about 12 wt. %, including ranges and sub-ranges there between, based on the total weight of the composition. Additionally or alternatively, the amount of ascorbic acid may be 2.0 wt. % to 20 wt. %, 2.5 wt. % to 20 wt. %, 3.5 wt. % to 20 wt. %, 4.5 wt. % to 20 wt. %, 5 wt. % to 15 wt. %, 6 wt. % to 14 wt. %, 7 wt. % to 13 wt. %, or 8 wt. % to 12 wt. % including ranges and sub-ranges there between, based on the total weight of the composition. Likewise, in some embodiments, the cosmetic composition may include an amount of ascorbic acid that is about 20 wt. %, about 19 wt. %, about 18 wt. %, about 17 wt. %, about 16 wt. %, about 15 wt. %, about 14 wt. %, about 13 wt. %, about 12 wt. %, about 11 wt. %, about 10 wt. %, about 9 wt. %, about 8 wt. %, about 7 wt. %, about 6 wt. %, about 5 wt. %, about 4 wt. %, about 3 wt. %, about 2 wt. % or about 1 wt. %, based on the total weight of the composition. In one embodiment, the amount of ascorbic acid may be 10% or about 10% of the total weight of the cosmetic composition.

The ascorbic acid may be L-ascorbic acid, a mixture of L-ascorbic acid and R-ascorbic acid, and/or a racemic mixture. In one embodiment, the ascorbic acid comprises only L-ascorbic acid, essentially only L-ascorbic acid, and/or at least 75 wt. %, at least 85 wt. %, at least 90 wt. %, at least 95 wt. %, or at least 99 wt. % of L-ascorbic acid based on the total weight of ascorbic acid.

Di-t-butyl Pentaerythrityl Tetrahydroxycinnamate

The cosmetic compositions include di-t-butyl pentaerythrityl tetrahydroxycinnamate.

Di-t-butyl pentaerythrityl tetrahydroxycinnamate or tetra-di-t-butyl pentaerythrityl hydroxyhydrocinnamate is a compound which belongs to the family of cinnamic acids and derivatives thereof, the CAS number of which is 6683-19-8.

By way of example, mention may be made of the di-t-butyl pentaerythrityl tetrahydroxycinnamate compound sold by the company BASF under the name Tinogard TT®.

Preferably, a composition according to the invention comprises from 0.05% to 0.5% by weight, preferably from 0.06% to 0.4% by weight and more preferentially from 0.8% to 0.3% by weight of di-t-butyl pentaerythrityl tetrahydroxycinnamate, relative to the total weight of the composition.

Hydroxyacetophenone

The cosmetic composition includes hydroxyacetophenone.

More particularly, the cosmetic composition of the present invention comprises hydroxyacetophenone or p-hydroxyacetophenone, which CAS number is 99-93-4 and also called 1-(4-hydroxyphenyl)-ethanone.

The hydroxyacetophenone has the following formula (II):

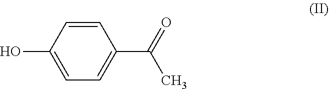

Mention may be made of the product sold under the name Symsave® H by the company Symrise.

In some instances, the total amount of hydroxyacetophenone may range from about 0.1, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.35, about 0.4, about 0.45, about 0.5, about 0.55, about 0.6, about 0.65, about 0.7, about 0.75, about 0.8, about 0.85, about 0.9, about 0.95, about 1.2, about 1.4, about 1.5 to about 1.5, about 1.6, about 1.65, about 1.7, about 1.75, about 1.8, about 1.85, about 1.9, about 1.95, about 2, about 2.1, about 2.2, about 2.3, about 2.5, about 2.5, about 2.6, about 2.7, about 2.8, about 2.9, or about 3 wt. %, including ranges and sub-ranges there between, based on the total weight of the composition. Additionally or alternatively, the total amount of hydroxyacetophenone may be from 0.1, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.5, 0.55, 0.6, 0.65, 0.7, 0.75, 0.8, 0.85, 0.9, 0.95, 1.2, 1.4, 1.5 to about 1.5, 1.6, 1.65, 1.7, 1.75, 1.8, 1.85, 1.9, 1.95, 2, 2.1, 2.2, 2.3, 2.5, 2.5, 2.6 2.7, 2.8, 2.9, or 3 wt. % including ranges and sub-ranges there between, based on the total weight of the composition.

Chelating Agents

The cosmetic compositions include one or more chelating agents. For example, the one or more chelating agents may include or be chosen from Trisodium Ethylenediamine Disuccinate, Sodium Phytate, Tetrasodium Glutamate Diacetate, and mixture thereof. In one instance, the one or more chelating agents comprises Trisodium Ethylenediamine Disuccinate and optionally one or more additional chelating agents. In another instance, the one or more chelating agents includes only Trisodium Ethylenediamine Disuccinate or essentially only Trisodium Ethylenediamine Disuccinate.

By way of example, mention may be made of the Trisodium Ethylenediamine Disuccinate compound sold under the name Natrlquest® E30 by the company Innospec Active Chemicals (at 37% by weight in water), or the compound sold under the name Octaquest E30® by the company Octel Performance Chemicals.

The total amount of chelating agents present in the cosmetic composition may be, e.g., about 0.01, about 0.02, about 0.03, about 0.04, about 0.05, about 0.06, about 0.07, about 0.08, about 0.09, about 0.1, about 0.15, about 0.2, about 0.25, about 0.3, about 0.35, about 0.4, about 0.45, about 0.5 to about 0.5, about 0.52, about 0.54, about 0.56, about 0.58, about 0.6, about 0.62, about 0.64, about 0.66, about 0.68, about 0.7, about 0.75, about 0.80, about 0.85, about 0.9, about 0.95, or about 1 wt. %; including ranges and sub-ranges there between, based on the total weight of the composition. Additionally or alternatively, the total amount of hydroxyacetophenone may be from 0.01, 0.02, 0.03, 0.04, 0.05, 0.06, 0.07, 0.08, 0.09, 0.1, 0.15, 0.2, 0.25, 0.3, 0.35, 0.4, 0.45, 0.5 to 0.5, 0.52, 0.54, 0.56, 0.58, 0.6, 0.62, 0.64, 0.66, 0.68, 0.7, 0.75, 0.80, about 0.85, 0.9, 0.95, or 1 wt. %; including ranges and sub-ranges there between, based on the total weight of the composition.

Non-limiting examples of chemical chelating agents include aminotrimethyl phosphonic acid, ß-alanine diacetic acid, citric acid, cyclodextrin, cyclohexanediamine tetracetic acid, diethylenetriamine pentamethylene phosphonic acid, diethanolamine N-acetic acid, ethylene diamine tetracetic acid (EDTA or YH4) and its sodium (YH3Na, Y2H2Na2, YHNa3 and YNa4), potassium (YH3K, Y2H3K3 and YK4), calcium disodium, and diammonium salts and its salts with triethanolamine (TEA-EDTA), etidronic acid, galactanic acid, hydroxyethyl ethylenediamine tetracetic acid (HEDTA) and its trisodium salt, gluconic acid, glucuronic acid, nitrilotriacetic acid (NTA) and its trisodium salt, pentetic acid, phytic acid, ribonic acid, diammonium citrate, disodium azacycloheptane diphosphonate, disodium pyrophoshate, hydroxypropyl cyclodextrin, methyl cyclodextrin, pentapotassium triphosphate, pentasodium aminotrimethylene phosphonate, pentasodium ethylenediamine tetramethylene phosphonate, pentasodium pentetate, pentasodium triphosphate, potassium citrate, potassium EDTMP, sodium EDTMP, sodium chitosan methylene phosphonate, sodium hexametaphosphate, sodium metaphosphate, potassium polyphosphate, sodium polyphosphate, sodium trimetaphosphate, sodium dihydroxyethylglycinate, potassium gluconate, sodium gluconate, sodium glucopeptate, sodium glycereth-1 polyphosphate, tetrapotassium pyrophosphate, triethanolamine polyphosphate (TEA), tetrasodium pyrophosphate, trisodium phosphate, potassium triphosphonomethylamine oxide, sodium metasilicate, sodium phytate, sodium polydimethylglycinophenolsulfonate, tetrahydroxyethyl ethylene diamine, tetrahydroxypropyl ethylene diamine, tetrapotassium etidronate, tetrasodium etidronate, tetrasodium iminodisuccinate, trisodium ethylenediamine disuccinate, ethanolamine N,N-diacetic acid, disodium acetate, dimercaprol, deferoxamine, Zylox, and/or iron chelating agent disclosed and claimed in the international patent application WO 94/61338, which is incorporated herein in its entirety for all purposes. Examples of biological chelating agents include metallothionein, transferrin, calmodulin, and sodium chitosan methylene phosphonate.

The cosmetic composition includes trisodium ethylenediamine disuccinate typically in an amount from about 0.01 to about 1 wt. %, based on the total weight of the cosmetic composition.

The total amount of chelating agents may vary but is typically from about 0.01 to about 1 wt. %, based on the total weight of the composition.

Fatty Compounds

The cosmetic composition may include one or more fatty compounds, which may be liquid or solid at room temperature and at atmospheric pressure (25° C., 1 atm). Fatty compounds are typically organic compounds that are not soluble in water at normal temperature (25° C.) and at atmospheric pressure (750 mmHg) (solubility below 10%). In some instances, the solubility in water may be below 5%, below 1%, or below 0.1%.

The total amount of fatty compounds in the compositions may vary from, e.g., about 0.5 to about 20 wt. %, about 0.5 to about 10 wt. %, about 0.5 to about 8 wt. %, about 0.5 to 6 wt. %, from about 1 to about 25 wt. %, about 1 to about 20 wt. %, about 1 to about 15 wt. %, about 1 to about 10 wt. %, about 1 to about 8 wt. %, or about 1 to about 6 wt. %, from about 1.5 to about 25 wt. %, about 1.5 to about 20 wt. %, about 1.5 to about 15 wt. %, about 1.5 to about 10 wt. %, about 1.5 to about 8 wt. %, or about 1.5 to about 6 wt. %, from about 2 to about 25 wt. %, about 2 to about 20 wt. %, about 2 to about 15 wt. %, about 2 to about 10 wt. %, about 2 to about 8 wt. %, or about 2 to about 6 wt. %, from about 2.5 to about 25 wt. %, about 2.5 to about 20 wt. %, about 2.5 to about 15 wt. %, about 2.5 to about 10 wt. %, about 2.5 to about 8 wt. %, or about 2.5 to about 6 wt. %, from about 3 to about 25 wt. %, about 3 to about 20 wt. %, about 3 to about 15 wt. %, about 3 to about 10 wt. %, about 3 to about 8 wt. %, or about 3 to about 6 wt. %, from about 3.5 to about 25 wt. %, about 3.5 to about 20 wt. %, about 3.5 to about 15 wt. %, about 3.5 to about 10 wt. %, about 3.5 to about 8 wt. %, or about 3.5 to about 6 wt. %, including ranges and sub-ranges there between, based on the total weight of the composition. Additionally or alternatively, the total amount of fatty compounds may be from 0.5 to 20 wt. %, 0.5 to 15 wt. %, 0.5 to 10 wt. %, 0.5 to 8 wt. %, 0.5 to 6 wt. %, from 1 to 25 wt. %, 1 to 20 wt. %, 1 to 15 wt. %, 1 to 10 wt. %, 1 to 8 wt. %, or 1 to 6 wt. %, from 1.5 to 25 wt. %, 1.5 to 20 wt. %, 1.5 to 15 wt. %, 1.5 to 10 wt. %, 1.5 to 8 wt. %, or 1.5 to 6 wt. %, from 2 to 25 wt. %, 2 to 20 wt. %, 2 to 15 wt. %, 2 to 10 wt. %, 2 to 8 wt. %, or 2 to 6 wt. %, from 2.5 to 25 wt. %, 2.5 to 20 wt. %, 2.5 to 15 wt. %, 2.5 to 10 wt. %, 2.5 to 8 wt. %, or 2.5 to 6 wt. %, from 3 to 25 wt. %, 3 to 20 wt. %, 3 to 15 wt. %, 3 to 10 wt. %, 3 to 8 wt. %, or 3 to 6 wt. %, from 3.5 to 25 wt. %, 3.5 to 20 wt. %, 3.5 to 15 wt. %, 3.5 to 10 wt. %, 3.5 to 8 wt. %, or 3.5 to 6 wt. %, including ranges and sub-ranges there between, based on the total weight of the composition.

Non-limiting examples of fatty compounds of the cosmetic composition include or may be chosen from oils, mineral oil, fatty alcohols, fatty acids, fatty alcohol derivatives, fatty acid derivatives (e.g., alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), glyceryl esters (glycerol esters), alkyl ethers of fatty alcohols, fatty acid esters of alkyl ethers of fatty alcohols, fatty acid esters of alkoxylated fatty alcohols, fatty acid esters of alkyl ethers of alkoxylated fatty alcohols, esters of fatty alcohols, hydroxy-substituted fatty acids, waxes, triglyceride compounds, lanolin, and a mixture thereof. In some instances, the one or more fatty compound may comprise or be chosen from fatty alcohols, fatty acids, esters of fatty acids, and/or esters of fatty alcohols (e.g., cetyl palmitate, cetyl stearate, myristyl myristate, myristyl stearate, cetyl myristate, and stearyl stearate (a mixture of which is referred to as “cetyl esters”)). Additionally or alternatively, the one or more fatty compounds may include or be chosen from hydrocarbons, fatty alcohols, fatty alcohol derivatives, fatty acids, fatty acid derivatives, fatty esters, fatty ethers, oils, waxes, etc. In one instance, the one or more fatty compounds is a hydrocarbon that is linear, branched, and/or cyclical, such as cyclic C₆-C₁₆ alkanes, hexane, undecane, dodecane, tridecane, and isoparaffins, for instance isohexadecane, isododecane and isodecane. Additionally, the linear or branched hydrocarbons may be composed only of carbon and hydrogen atoms of mineral, plant, animal or synthetic origin with more than 16 carbon atoms, such as volatile or non-volatile liquid paraffins, petroleum jelly, liquid petroleum jelly, polydecenes, hydrogenated polyisobutene, and squalane.

Fatty Alcohols

The one or more fatty compounds may be glycerolated and/or oxyalkylenated, may include from 8 to 30 carbon atoms, and may be saturated or unsaturated. The fatty alcohols useful herein include those having from about 8 to about 30 carbon atoms, from about 12 to about 22 carbon atoms, and from about 14 to about 22 carbon atoms. These fatty alcohols can be straight or branched chain alcohols and can be saturated or unsaturated. Nonlimiting examples of fatty alcohols include decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, cis4-t-butylcyclohexanol, myricyl alcohol and a mixture thereof. In some cases, the fatty alcohols comprise at least one of or may be chosen from myristyl alcohol, cetyl alcohol, stearyl alcohol, isostearyl alcohol, oleyl alcohol, isotridecyl alcohol, and a mixture thereof.

The saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

The unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

Non-limiting examples of solid fatty alcohols include linear or branched, saturated or unsaturated alcohols containing from 8 to 30 carbon atoms, for example, myristyl alcohol, cetyl alcohol, stearyl alcohol and their mixture, cetylstearyl alcohol.

Fatty Esters

The fatty compounds of the cosmetic composition may be liquid or solid fatty esters at 25° C., 1 atm. The fatty esters may include esters from a C₆-C₃₂ fatty acid and/or a C₆-C₃₂ fatty alcohol. For example, the fatty compounds may include or be chosen from fatty acid monoesters and diesters, polyol esters, polyglycerol esters, polyglycerol polyricinoleate, polyglycerol poly-12-hydroxystearate, polyglycerol dimerate isostearate, ethylhexanoate, polyglycerol esters, and a mixture thereof. These esters may be esters of saturated or unsaturated, linear or branched C₁-C₂₆ aliphatic mono or polyacids and of saturated or unsaturated, linear or branched C₁-C₂₅ aliphatic mono or polyalcohols, the total number of carbon atoms in the esters being greater than or equal to 10. In one instance, the fatty compounds comprise one or more fatty acid monoesters. For the esters of monoalcohols, at least one of the alcohol or the acid from which the esters result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate and isostearyl neopentanoate.

In some instances, the fatty esters are cetyl esters, such as esters of saturated fatty acids and fatty alcohols. For example, the fatty esters may include or be chosen from cetyl palmitate, cetyl stearate, myristyl myristate, myristyl stearate, cetyl myristate, stearyl stearate, cetearyl ethylhexanoate, and mixtures thereof. In one instance, the fatty esters may be one or more of or chosen from isopropyl isostearate, n-propyl myristate, isopropyl myristate, hexyl laurate, hexadecyl isostearate, hexydecyl laurate, hexyldecyl octanoate, n-propyl palmitate, isopropyl palmitate, and mixtures thereof. In another instance, the fatty esters include or may be chosen from diisobutyl adipate, 2-hexyldecyl adipate, di-2-heptylundecyl adipate, monoisostearic acid N-alkyl glycol, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, isocetyl stearate, butyl stearate, diisopropyl sebacate, di-2-ethylhexyl sebacate, cetyl lactate, myristyl lactate, isopropyl palm itate, 2-ethylhexyl palm itate, 2-hexyldecyl palm itate, 2-heptylundecyl palm itate, cholesteryl 12-hydroxystearate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, diisostearyl malate, dicaprylyl carbonate, cetearyl ethylhexanoate, and mixtures thereof. In yet a further instance, the cosmetic composition includes one or more of or may have fatty compounds chosen from cetearyl alcohol, cetearyl ethylhexanoate, isopropyl myristate, and mixtures thereof.

Non-limiting examples of liquid fatty acid include triglyceride oils of plant or synthetic origin, such as liquid fatty acid triglycerides containing 6 to 30 carbon atoms, for instance heptanoic or octanoic acid triglycerides, or alternatively, e.g., sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camellina oil, tamanu oil, babassu oil and pracaxi oil, jojoba oil, shea butter oil, and mixtures thereof.

Non-limiting examples of solid fatty acid esters and/or fatty acid esters that may be mentioned include solid esters obtained from C₉-C₂₆ fatty acids and from C₉-C₂₅ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

Fatty Alcohol Derivatives

The cosmetic compositions may, in some instances, include fatty alcohol derivatives such as alkyl ethers of fatty alcohols, alkoxylated fatty alcohols, alkyl ethers of alkoxylated fatty alcohols, esters of fatty alcohols and a mixture thereof. Non-limiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof. Liquid fatty ethers may be chosen from liquid dialkyl ethers such as dicaprylyl ether. The non-liquid fatty ethers may also be chosen from dialkyl ethers and in particular dicetyl ether and distearyl ether, alone or as a mixture.

Esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate; diisopropyl sebacate; diisopropyl adipate; di-n-propyl adipate; triisopropyl citrate; glyceryl trilactate; glyceryl trioctanoate; neopentyl glycol diheptanoate; and diethylene glycol diisononanoate.

Fatty Acid Derivatives

The cosmetic compositions may, in some instances, include fatty acid derivatives. The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof.

The cosmetic composition may, in some cases, have 5 wt. % or less, 4 wt. % or less, 3 wt. % or less, 2 wt. % or less, or 1 wt. % or less of tocopherol. In at least one instance, the cosmetic composition is free of or essentially free of tocopherol.

The cosmetic compositions are generally formulated as an emulsion. Typically, the cosmetic compositions are formulated to be oil-in-water emulsions, with the fatty phase emulsified in the hydrophilic phase.

Emulsifiers

The cosmetic compositions described herein include one or more emulsifiers. For example, the emulsifier may be an amphoteric, anionic, cationic or nonionic emulsifier, used alone or as a mixture, and optionally with a co-emulsifier. The emulsifiers are chosen in an appropriate manner according to the emulsion to be obtained.

For O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

The one or more emulsifiers may be oxyalkylenated organosiloxane emulsifiers. The oxyalkylenated organosiloxane emulsifiers may be fully or partially crosslinked and/or be elastomeric or non-elastomeric. They are sometimes referred to as “emulsifying elastomers” because of they have both elastomeric and emulsifying properties. In some instances, the one or more emulsifiers include an oganosiloxane emulsifier, including crosslinked organosiloxane emulsifiers. For example, the cosmetic compositions may comprise one or more crosslinked organosiloxane emulsifier including or chosen from dimethicone/dimethicone PEG/PPG 15 crosspolymer, dimethicone PEG-10 crosspolymer, dimethicone PEG-10/15 crosspolymer, dimethicone PEG-15 crosspolymer, dimethicone polyglycerin-3 crosspolymer, dimethicone PPG-20 crosspolymer, dimethiconol/methylsilanol/silicate crosspolymer; dimethiconol/silicate crosspolymer, lauryl dimethicone PEG-15 crosspolymer, lauryl dimethicone polyglycerin-3 crosspolymer, PEG-8 dimethicone polysorbate-20 crosspolymer, PEG-10 dimethicone/vinyl dimethicone crosspolymer, PEG-10 lauryl dimethicone crosspolymer, PEG-15/lauryl dimethicone crosspolymer, PEG-15 laurylpolydimethylsiloxyethyl crosspolymer, and mixtures thereof.

In another instance, the cosmetic compositions include one or more linear organosiloxane emulsifier chosen from cyclotetrasiloxane (and) cyclopentasiloxane (and) PEG/PPG-18 dimethicone, cyclopentasiloxane (and) PEG/PPG-18/18 dimethicone; PEG/PPG-18/18 dimethicone; lauryl PEG/PPG-18/18 methicone; cetyl PEG/PPG-14/14 dimethicone; bis-cetyl PEG/PPG-14/14 dimethicone; cetyl PEG/PPG-10/1 dimethicone; PEG-11 methyl ether dimethicone; PEG/PPG-20/22 butyl ether dimethicone; PEG-9 dimethicone; PEG-3 dimethicone; PEG-9 methyl ether dimethicone; PEG-10 dimethicone; lauryl PEG-9 polydimethylsiloxyethyl dimethicone; and mixtures thereof.

Further examples of organosiloxane emulsifiers include those having C.T.F.A. names Bis-Butyldimethicone Polyglyceryl-3; Bis-PEG/PPG-14/14 Dimethicone; Bis-butyldimethicone Polyglyceryl-3; Bis-isobutyl PEG/PPG-10/7 Dimethicone copolymer; Bis-PEG/PPG-18/6 Dimethicone; Bis-PEG/PPG-20/20 Dimethicone; Bis-PEG/PPG-16/16 PEG/PPG-16/16 Dimethicone; Bis(PPG-7 Undeceneth-21-Dimethicone; Cetyl Dimethicone PEG-7 Acetate; Cetyl PEG-8 Dimethicone; Cetyl PEG/PPG-15/16 Butyl Ether Dimethicone; Cetyl PEG/PPG-15/15 Butyl Ether Dimethicone; Cetyl PEG/PPG-7/3 Dimethicone; Cetyl PEG/PPG-10/1 Dimethicone; Dimethicone PEG-15 Acetate; Dimethicone PEG-7 Cocoate; Dimethicone PEG-7 Phosphate; Dimethicone PEG-10 Phosphate; Dimethicone PEG/PPG-7/4 Phosphate; Dimethicone PEG/PPG-12/4 Phosphate; Dimethicone PEG-7 Undecylenate; Lauryl Dimethicone PEG-10 Phosphate; Isopolyglyceryl-3 Dimethicone; Isopolyglyceryl-3 Dimethiconol; Isostearyl Carboxyldecyl PEG-8 Dimethicone; Lauryl Methicone PEG-10 Phosphate; Lauryl PEG-8 Dimethicone; Lauryl PEG-10 Methyl Ether Dimethicone; Lauryl PEG/PPG-18/18 Methicone; PEG-6 Methyl Ether Dimethicone; PEG-7 Methyl Ether Dimethicone; PEG-9 Methyl Ether Dimethicone; PEG-10 Methyl Ether Dimethicone; PEG-11 Methyl Ether Dimethicone; PEG-11 Methyl Ether Dimethicone; PEG-32 Methyl Ether Dimethicone; PEG-PEG/PPG-28/21 Acetate Dimethicone; PEG/PPG-22/22 Butyl Ether Dimethicone; PEG/PPG-23/23 Butyl Ether Dimethicone; PEG/PPG-24/18 Butyl Ether Dimethicone; PEG/PPG-3/10 Dimethicone; PEG/PPG-4/12 Dimethicone; PEG/PPG-6/11 Dimethicone; PEG/PPG-8/14 Dimethicone; PEG/PPG-12/16 Dimethicone; PEG/PPG-12/18 Dimethicone; PEG/PPG-14/4 Dimethicone; PEG/PPG-15/5 Dimethicone; PEG/PPG-15/15 Dimethicone; PEG/PPG-16/2 Dimethicone; PEG/PPG-16/8 Dimethicone; PEG/PPG-17/18 Dimethicone; PEG/PPG-18/12 Dimethicone; PEG/PPG-19/19 Dimethicone; PEG/PPG-20/6 Dimethicone; PEG/PPG-20/15 Dimethicone; PEG/PPG-20/20 Dimethicone; PEG/PPG-20/29 Dimethicone; PEG/PPG-22/23 Dimethicone; PEG/PPG-22/24 Dimethicone; PEG/PPG-25/25 Dimethicone; PEG/PPG-27/27 Dimethicone; PEG/PPG-30/10 Dimethicone; PEG/PPG-10/3 Oleyl Ether Dimethicone; PEG-8 trisiloxane; Polyglyceryl-3 Polydimethylsiloxyethyl Dimethicone; PPG-12 Butyl Ether Dimethicone; Silicone Quaternium-17; TEA-Dimethicone PEG-7 Phosphate; and mixtures thereof.

Further examples of commercial linear organosiloxane emulsifiers are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Further examples of crosslinked organosiloxane emulsifiers include, but are not limited to Dimethicone/dimethicone PEG/PPG 15 crosspolymer; Dimethicone PEG-10 crosspolymer; Dimethicone PEG-10/15 Crosspolymer; Dimethicone PEG-15 Crosspolymer; Dimethicone Polyglycerin-3 Crosspolymer; Dimethicone PPG-20 Crosspolymer; Dimethiconol/Methylsilanol/Silicate Crosspolymer; Dimethiconol/Silicate Crosspolymer; Lauryl Dimethicone PEG-15 Crosspolymer; Lauryl Dimethicone Polyglycerin-3 Crosspolymer; PEG-8 Dimethicone Polysorbate-20 Crosspolymer; PEG-10 Dimethicone/Vinyl Dimethicone Crosspolymer; PEG-10 Lauryl Dimethicone Crosspolymer; PEG-15/Lauryl Dimethicone Crosspolymer; and PEG-15 Laurylpolydimethylsiloxyethyl Crosspolymer.

The emulsifiers may, in some instances, be nonionic a surfactant, such as one chosen from: alkanolamides; alkyl polyglucosides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

Additional nonionic surfactants that may, in some instances, be suitable include, e.g., alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C6-C24)alkylpolyglycosides; N—(C₆-C₂₄)alkylglucamine derivatives, amine oxides such as (C10-C14)alkylamine oxides or N—(C₁₀-C₁₄)acylaminopropylmorpholine oxides; and mixtures thereof.

In some cases, the nonionic surfactant may be chosen from esters of polyols with fatty acids with a saturated or unsaturated chain containing for example from 8 to 24 carbon atoms, and alkoxylated derivatives thereof; polyethylene glycol esters of a C₈-C₂₄; sorbitol esters of a C₈-C₂₄; sugar (sucrose, glucose, alkylglycose) esters of a C₈-C₂₄, preferably C₁₂-C₂₂, fatty acid or acids and alkoxylated derivatives thereof; ethers of sugar and a C₈-C₂₄, preferably C₁₂-C₂₂, fatty alcohol or alcohols; and mixtures thereof. In one instance, the nonionic surfactant is an ethoxylated fatty ester chosen from adducts of ethylene oxide with esters of lauric acid, palmitic acid, stearic acid or behenic acid, and mixtures thereof. Examples of ethoxylated fatty esters that may be suitable include those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

The nonionic surfactant may be chosen from glyceryl esters of fatty acids, glyceryl stearate (glyceryl mono-, di- and/or tristearate) (CTFA name: glyceryl stearate); glyceryl ricinoleate; glyceryl esters of C₈-C₂₄ alkoxylated fatty acids, such as polyethoxylated glyceryl stearate (glyceryl mono-, di- and/or tristearate), and PEG-20 glyceryl stearate; and mixtures thereof.

In some instances, the cosmetic composition may include an emulsifier such as dimers surfactants named “gemini surfactants,” which may have two surfactant moieties identical or different, and constituted by a hydrophilic head group and a lipophilic group linked to each other through the head groups, thanks to a spacer. For example, the one or more emulsifiers may include or be chosen from those sold by Sasol company under the name CERALUTIOM, for example, CERALUTION H: Behenyl Alcohol, Glyceryl Stearate, Glyceryl Stearate Citrate et Sodium Dicocoyl ethylenediamine PEG-15 Sulfate, CERALUTION F: Sodium Lauroyl Lactylate et Sodium Dicocoyl ethylenediamine PEG-15 Sulfate, CERALUTION C: Aqua, Capric/Caprylic triglyceride, Ceteareth-25, Sodium Dicocoyl ethylenediamine PEG-15 Sulfate, Sodium Lauroyl Lactylate, Behenyl Alcohol, Glyceryl Stearate, Glyceryl Stearate Citrate, Gum Arabic, Xanthan Gum, Phenoxyethanol, Methylparaben, Ethylparaben, Butylparaben, Isobutylparaben. In one embodiment, the emulsifier of the cosmetic composition consists of sodium lauroyl lactylate or consists essentially of sodium lauroyl lactylate. In another embodiment, the emulsifier(s) of the cosmetic composition includes sodium lauroyl lactylate with one or more additional emulsifiers, such as a nonionic emulsifier or an anionic emulsifier.

The total amount of emulsifiers in the compositions may vary from, e.g., about 0.001 to about 25 wt. %, based on the total weight of the composition. For example, the total amount of fatty compounds may be from about 0.001 to about 25 wt. %, about 0.001 to about 20 wt. %, from about 0.001 to about 15 wt. %, about 0.001 to about 10 wt. %, about 0.001 to about 8 wt. %, about 0.001 to about 6 wt. %, from about 0.005 to about 25 wt. % about 0.005 to about 20 wt. %, about 0.005 to about 15 wt. %, about 0.005 to about 10 wt. %, about 0.005 to about 8 wt. %, about 0.005 to 6 wt. %, from about 0.01 to about 25 wt. %, about 0.01 to about 20 wt. %, about 0.01 to about 15 wt. %, about 0.01 to about 10 wt. %, about 0.01 to about 8 wt. %, or about 0.01 to about 6 wt. %, from about 0.05 to about 25 wt. %, about 0.05 to about 20 wt. %, about 0.05 to about 15 wt. %, about 0.05 to about 10 wt. %, about 0.05 to about 8 wt. %, or about 0.05 to about 6 wt. % including ranges and sub-ranges there between, based on the total weight of the composition. In one instance, the total amount of emulsifiers in the cosmetic composition are typically in an amount from 0.5, 0.6, 0.7, 0.8, 0.9 or 1.0 wt. % to 5, 6, 7, 8, 9, or 10 wt. %.

Cationic Surfactant

The cosmetic compositions may optionally include one or more cationic surfactants. As used herein, the term “cationic surfactant” refers to a surfactant that may be positively charged when it is contained in the cosmetic compositions according to the disclosure. This surfactant may bear one or more positive permanent charges or may contain one or more functional groups that are cationizable in the cosmetic composition according to the disclosure. Non-limiting examples of cationic surfactants that may be in the cosmetic composition include or may be chosen from behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, and a mixture thereof.

In one instance, the cationic surfactants include or may be chosen from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and a mixture thereof.

The cationic surfactant(s) may optionally be chosen from polyoxyalkylenated, primary, secondary or tertiary fatty amines, or salts thereof, and quaternary ammonium salts, and mixtures thereof. In some cases, it is useful to use salts such as chloride salts of the quaternary ammonium compounds. The quaternary ammonium compounds may be fatty amines having, e.g., at least one C₈-C₃₀ hydrocarbon-based chain. Examples of quaternary ammonium salts include those having a structure in accordance with the general formula provided below:

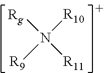

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Exemplary quaternary ammonium salts having a structuring in accordance with the above formula include tetraalkylammonium salts (e.g., dialkyldimethylammonium or alkyltrimethylammonium salts in which the alkyl group contains approximately from 12 to 22 carbon atoms, behenyltrimethylammonium, distearyldimethylammonium, cetyltrimethylammonium or benzyldimethylstearylammonium salts), oleocetyldimethylhydroxyethylammonium salts, palmitylamidopropyltrimethylammonium salts, stearamidopropyltrimethylammonium salts and stearamidopropyldimethylcetearylammonium salts.

Additional examples of quaternary ammonium salts include quaternary ammonium salt of imidazoline, such as those having a structure in accordance with the general formula provided below:

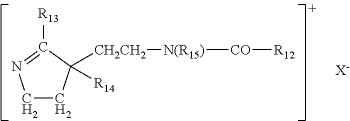

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms. R₁₂ and R₁₃ preferably denote a mixture of alkenyl or alkyl groups containing from 12 to 21 carbon atoms, derived for example from tallow fatty acids, R₁₄ preferably denotes a methyl group, and R₁₅ preferably denotes a hydrogen atom.

Additional examples of quaternary ammonium salts include quaternary diammonium or triammonium salt, such as those having a structure in accordance with the general formula provided below:

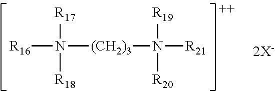

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75),

In some instances, the cationic surfactant is preferably selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof. In other instances, the cationic surfactants are more preferably selected from cetrimonium chloride, behentrimonium chloride, behentrimonium methosulfate, stearamidopropyl dimethylamine, and a mixture thereof. In one instance, the cosmetic composition includes solely behentrimonium methosulfate or essentially solely behentrimonium methosulfate. In another instance, however, the cosmetic composition includes behentrimonium methosulfate and at least another cationic surfactant, such as, e.g., cetrimonium chloride, behentrimonium chloride, or a mixture thereof.

The cosmetic composition may include an amount of cationic surfactants that is greater than zero to about 15 wt. %, based on the total weight of the composition. For example, the total amount of cationic surfactant(s) may be from greater than zero to about 14 wt. %, greater than zero to about 12 wt. %, greater than zero to about 10 wt. %, greater than zero to about 8 wt. %, greater than zero to about 6 wt. %, greater than zero to about 5 wt. %, greater than zero to about 4 wt. %, greater than zero to about 3 wt. %, greater than zero to about 2 wt. %, greater than zero to about 1 wt. %, from about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, from about 0.5 to about 10 wt. %, about 0.5 to about 8 wt. %, about 0.5 to about 5 wt. %, about 0.5 to about 4 wt. %, about 0.5 to about 3 wt. %, about 0.5 to about 2 wt. %, about 0.5 to about 1 wt. %, from about 1 to about 10 wt. %, about 1 to about 8 wt. %, about 1 to about 5 wt. %, about 1 to about 4 wt. %, or about 1 to about 3 wt. %, including ranges and sub-ranges there between, based on the total weight of the composition. Additionally or alternatively, the total amount of cationic surfactant(s) may be from greater than zero to 14 wt. %, greater than zero to 12 wt. %, greater than zero to 10 wt. %, greater than zero to 8 wt. %, greater than zero to 6 wt. %, greater than zero to 5 wt. %, greater than zero to 4 wt. %, greater than zero to 3 wt. %, greater than zero to 2 wt. %, greater than zero to 1 wt. %, from 0.1 to 8 wt. %, 0.1 to 6 wt. %, 0.1 to 5 wt. %, 0.1 to 4 wt. %, 0.1 to 3 wt. %, 0.1 to 2 wt. %, 0.1 to 1 wt. %, from 0.5 to 10 wt. %, 0.5 to 8 wt. %, 0.5 to 5 wt. %, 0.5 to 4 wt. %, 0.5 to 3 wt. %, 0.5 to 2 wt. %, 0.5 to 1 wt. %, from 1 to 10 wt. %, 1 to 8 wt. %, 1 to 5 wt. %, 1 to 4 wt. %, or 1 to 3 wt. %, including ranges and sub-ranges there between, based on the total weight of the cosmetic composition.

Thickening Agent(s)

The cosmetic compositions described herein may, optionally, include one or more thickening agents. The amount of thickening agents may depend on the other components in cosmetic composition and desired viscosity for the cosmetic composition.

The thickening agent may be in an amount of about 0.05 wt. % to about 5 wt. %, about 0.05 wt. % to about 4 wt. %, about 0.05 wt. % to about 3 wt. %, about 0.05 wt. % to about 2 wt. %, about 0.05 wt. % to about 1 wt. %; about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %; about 0.2 wt. % to about 5 wt. %, about 0.2 wt. % to about 4 wt. %, about 0.2 wt. % to about 3 wt. %, about 0.2 wt. % to about 2 wt. %, about 0.2 wt. % to about 1 wt. %; about 0.3 wt. % to about 5 wt. %, about 0.3 wt. % to about 4 wt. %, about 0.3 wt. % to about 3 wt. %, about 0.3 wt. % to about 2 wt. %, or about 0.3 wt. % to about 1 wt. %, including ranges and subranges thereof, based on the total weight of the cosmetic composition. Further, the amount of thickening agent may be from 0.05, 0.1, 0.2, 0.3, 0.4, 0.5, 0.6, 0.7, 0.8, 0.9, 1.0, or 1.5 wt. % to 2, 3, 4, or 5 wt. %, including ranges and subranges thereof, based on the total weight of the cosmetic composition.

Many thickening agents are water-soluble, and increase the viscosity of water or form an aqueous gel when the cosmetic composition of the invention is dispersed/dissolved in water. The thickening agent other than sodium polyacrylate may be dispersed/dissolved in an aqueous solvent that is soluble in water, e.g., ethyl alcohol when it is dispersed/dissolved in water.

Thickening agents may be referred to as “thickeners” or “viscosity modifying agents.” Thickening agents are typically included to increase the viscosity of the compositions. Nonetheless, in some instances, certain thickening agents provide additional, surprising benefits to the compositions. Non-limiting examples of thickening agents include polyacrylate crosspolymers (other than sodium polyacrylate) or crosslinked polyacrylate polymers (other than crosslinked sodium polyacrylate), cationic acrylate copolymers, anionic acrylic or carboxylic acid polymers, polyacrylamide polymers, polysaccharides such as cellulose derivatives, gums, polyquaterniums, vinylpyrrolidone homopolymers/copolymers, C8-24 hydroxyl substituted aliphatic acid, C8-24 conjugated aliphatic acid, sugar fatty esters, polyglyceryl esters, and a mixture thereof. Suitable thickeners other than sodium polyacrylate may be found in U.S. patent application Ser. No. 16/731,654, which is incorporated herein, in its entirety for all purposes.

The thickening agents may be chosen from xanthan gum, guar gum, biosaccharide gum, cellulose, acacia seneca gum, sclerotium gum, agarose, pechtin, gellan gum, hyaluronic acid. Additionally, the thickening agents may include polymeric thickeners selected from the group consisting of ammonium polyacryloyldimethyl taurate, ammonium acryloyldimethyltaurate/VP copolymer, acrylates copolymers, polyacrylamide, carbomer, and acrylates/C10-30 alkyl acrylate crosspolymer.

Particular types of thickening agents that may be mentioned include the following:

Carboxylic Acid or Carboxylate Based Homopolymer or Co-Polymer, which can be Linear or Crosslinked:

These polymers contain one or more monomers derived from acrylic acid, substituted acrylic acids, and salts and esters of these acrylic acids (acrylates) and the substituted acrylic acids. Commercially available polymers include those sold under the trade names Carbopol, Acrysol, Polygel, Sokalan, Carbopol Ultrez, and Polygel. Examples of commercially available carboxylic acid polymers include the carbomers, which are homopolymers of acrylic acid crosslinked with allyl ethers of sucrose or pentaerytritol. The carbomers are available as the Carbopol 900 series from B.F. Goodrich (e.g., Carbopol 954). In addition, other suitable carboxylic acid polymeric agents include Ultrez 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol 1342, Carbopol 1382, Pemulen TR-1, and Pemulen TR-2, from B.F. Goodrich.

Other suitable carboxylic acid or carboxylate polymeric agents include copolymers of acrylic acid and alkyl C5-C10 acrylate, copolymers of acrylic acid and maleic anhydride, and polyacrylate crosspolymer-6. Polyacrylate Crosspolymer-6 is aviable in the raw material known as SEPIMAX ZEN from Seppic.

Another suitable carboxylic acid or carboxylate polymeric agent includes acrylamidopropyltrimonium chloride/acrylates copolymer, a cationic acrylates copolymer (or a quaternary ammonium compound), available as a raw material known under the tradename of SIMULQUAT HC 305 from Seppic.

In certain embodiments, the carboxylic acid or carboxylate polymer thickeners useful herein are those selected from carbomers, acrylates/C10-C30 alkyl acrylate crosspolymers, polyacrylate crosspolymer-6, acrylamidopropyltrimonium chloride/acrylates copolymer, and mixtures thereof.

Polyquaternium Compounds

Non-limiting examples, include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof. In some instances, the cosmetic compositions include polyquaternium-10, polyquaternium-11, polyquaternium-67, or a mixture thereof.

Celluloses

Non-limiting examples of celluloses include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. In some instances, the cellulose is selected from water-soluble cellulose derivatives (for example, carboxymethyl cellulose, methyl cellulose, methylhydroxypropyl cellulose, hydroxyethyl cellulose, hydroxypropyl cellulose, cellulose sulfate sodium salt). Furthermore, in some instance, the cellulose is preferably hydroxypropylcellulose (HPC). In some instances, the cosmetic compositions include one or more cellulose thickeners (e.g., microcrystalline cellulose, carboxymethylcellulose, hydroxymethylcellulose, and hydroxypropylcellulose).

Polyvinylpyrrolidone (PVP) and Co-Polymers

Non-limiting examples include Polyvinylpyrrolidone (PVP), Polyvinylpyrrolidone (PVP)/vinyl acetate copolymer (PVP/VA copolymer), polyvinylpyrrolidone (PVP)/eicosene copolymer, PVP/hexadecene copolymer, etc. Commercially available polyvinylpyrrolidone includes Luviskol K30, K85, K90 available from BASF. Commercially available copolymers of vinylpyrrolidone and vinylacetate include Luviskol VA37, VA64 available from BASF; copolymers of vinylpyrrolidone, methacrylamide, and vinylimidazole (INCI: VP/Methacrylamide/Vinyl Imidazole Copolymer) is commercially available as Luviset from BASF. In some instances, PVP and PVP/VA copolymer are preferred.

Sucrose Esters

Non-limiting examples include sucrose palmitate, sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate, and mixtures thereof.

Polyglyceryl Esters

Non-limiting polyglycerol esters of fatty acids (polygylceryl esters) include those of the following formula:

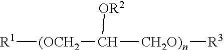

wherein n is from 2 to 20 or from 2 to 10 or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.

C8-24 Hydroxyl Substituted Aliphatic Acid and C8-24 Conjugated Aliphatic Acid

Non-limiting examples include conjugated linoleic acid, cis-parinaric acid, trans-7-octadecenoic acid, cis-5,8,11,14,17-eicosapentanoic acid, cis-4,7,10,13,16,19-docosahexenoic acid, columbinic acid, linolenelaidic acid, ricinolaidic acid, stearidonic acid, 2-hydroxystearic acid, alpha-linolenic acid, arachidonic acid, cis-11,14-eicosadienoic acid, linolelaidic acid, monopetroselinic acid, petroselinic acid, ricinoleic acid, trans-vaccenic acid, cis-11,14,17-eicosatrienoic acid, cis-5-eicosenoic acid, cis-8,11,14-eicosatrienoic acid, hexadecatrienoic acid, palmitoleic acid, petroselaidic acid, trans farnesol, cis-13,16-docosadienoic acid, cis-vaccenic acid, cis-11-eicosenoic acid, cis-13,16,19-docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, pinolenic acid, trans-13-octadecenoic acid. More preferably, the aliphatic acid comprises 12-hydroxystearic acid, conjugated linoleic acid, or a mixture thereof.

Gums

Non-limiting examples of gums include gum arabic, tragacanth gum, karaya gum, guar gum, gellan gum, tara gum, locust bean gum, tamarind gum, xanthan gum, locust bean gum, seneca gum, sclerotium gum, etc.

Additional examples of water-soluble thickeners include water-soluble natural polymers, water-soluble synthetic polymers, clay minerals, and silicic anhydride. Non-limiting examples of water-soluble natural polymers include gum arabic, tragacanth gum, karaya gum, guar gum, gellan gum, tara gum, locust bean gum, tamarind gum, sodium alginate, alginic acid propyleneglycol ester, carrageenan, farcelluran, agar, high-methoxy pectin, low-methoxy pectin, xanthine, chitosan, starch (for example starch derived from corn, potato, wheat, rice, sweet potato and tapioca, a-starch, soluble starch), fermentation polysaccharide (for example, xanthan gum, pullulan, carciran, dextran), acidic hetero-polysaccharide derived from callus of plants belonging to Polyantes sp. (for example, tuberous polysaccharide), proteins (for example, sodium casein, gelatin, albumin), chondroitin sulfate, and hyaluronic acid.

The cosmetic composition may include water-soluble synthetic polymers including, e.g., polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and starch derivatives (for example, starch oxide, dialdehyde starch, dextrin, British gum, acetyl starch, starch phosphate, carboxymethyl starch, hydroxyethyl starch, hydroxypropyl starch).

Polyol(s)

The cosmetic compositions may include one or more polyol(s). The amount of polyol in the cosmetic composition, if present, may range from about 1 to about 30 wt. %, based on the total weight of the cosmetic composition. For example, the cosmetic composition may include water-soluble solvents in an amount of about 1 to about 30 wt. %, about 1 to about 25 wt. %, about 1 to about 20 wt. %, about 1 to about 18 wt. %, about 1 to about 16 wt. %, about 1 to about 14 wt. %, about 1 to about 12 wt. %, about 1 to about 10 wt. %; about 5 to about 30 wt. %, about 5 to about 25 wt. %, about 5 to about 20 wt. %, about 5 to about 18 wt. %, about 5 to about 16 wt. %, about 5 to about 14 wt. %, about 5 to about 12 wt. %, about 5 to about 10 wt. %; about 10 to about 30 wt. %, about 10 to about 25 wt. %, about 10 to about 20 wt. %, about 10 to about 18 wt. %, about 10 to about 16 wt. %, about 10 to about 14 wt. %; about 12 to about 30 wt. %, about 12 to about 25 wt. %, about 12 to about 20 wt. %, about 12 to about 18 wt. %, about 12 to about 16 wt. %; about 14 to about 30 wt. %, about 14 to about 25 wt. %, about 14 to about 20 wt. %, about 14 to about 18 wt. %; about 16 to about 30 wt. %, about 16 to about 25 wt. %, about 16 to about 20 wt. %; about 18 to about 30 wt. %, about 18 to about 25 wt. %, about 18 to about 20 wt. %, including ranges and subranges thereof, based on the total weight of the cosmetic composition.

Polyhydric alcohols are useful. The term “polyol” should be understood as meaning, within the meaning of the present disclosure, an organic molecule comprising at least two free hydroxyl groups. The polyols of the cosmetic composition may be glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Non-limiting examples of polyols that may, optionally, be included in the cosmetic composition include and/or may be chosen from alkanediols such as glycerin, 1,2,6-hexanetriol, trimethylolpropane, ethylene glycol, propylene glycol, butylene glycol, pentylene glycol, hexylene glycol, dipropylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof. The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol.

In some cases, the polyol comprises glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In some cases, the polyol is chosen from ethylene glycol, propylene glycol, butylene glycol, hexylene glycol, pentylene glycol, diethylene glycol, dipropylene glycol, caprylyl glycol, 1,3 propanediol, glycerin, diglycerin, polyethylene glycols, and a mixture thereof.

Skin Active Agents

The cosmetic compositions may, optionally, include one or more skin active agents, such as anti-aging agent, anti-wrinkle actives, anti-oxidants, humectants, moisturizing ingredients, depigmenting agents, and/or agents for treating oily skin etc. The skin active agents may be included in the cosmetic composition in an amount ranging from greater than zero to about to about 5 wt. %, based on the total weight of the composition. For example, the total amount of skin active agents may be from greater than zero to about 5 wt. %, greater than zero to about 4 wt. %, greater than zero to about 3 wt. %, greater than zero to about 2 wt. %; about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.05 to about 5 wt. %, about 0.05 to about 2.5 wt. %, about 0.05 to about 1 wt. %, about 0.05 to about 0.5 wt. %; about 0.1 to about 5 wt. %, about 0.1 to about 2.5 wt. %, about 0.1 to about 1 wt. %, about 0.1 to about 0.5 wt. %; about 1 to about 5 wt. %, about 1 to about 4 wt. %, about 1 to about 3 wt. %; about 2 to about 5 wt. %, about 2 to about 4 wt. %; or about 3 to about 5 wt. %, including ranges and subranges there between, based on the total weight of the cosmetic composition.

A non-limiting discussion of skin active agents that may, in some cases, be included in the cosmetic composition is provided below:

Humectants and/or Moisturizing Ingredients

Examples of humectants and/or moisturizing ingredients include glycerol and its derivatives, urea and its derivatives, especially Hydrovance marketed by National Starch, hyaluronic acid, sodium pidolate, xylitol, serine, ectoin and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NutraLipids, a C-glycoside derivatives, in particular the C-13-D-xylopyranoside-2-hydroxypropane in the form of a solution at 30% by weight of active material in a water/propylene glycol mixture (60/40 wt %) as the product produced by the company Chimex under the trade name “Mexoryl SBB”, a rose hip oil marketed by Nestle, a micro-algae extract Prophyridium cruentum enriched with zinc, marketed under the name by Vincience Algualane Zinc spheres of collagen and chondroitin sulfate of marine origin (Atelocollagen) sold by the company Engelhard Lyon under the name Marine Filling Spheres, hyaluronic acid spheres such as those marketed by Engelhard Lyon, and arginine.

### Depigmenting Agents

Depigmenting agents that may be incorporated in the cosmetic composition include those chosen from alpha and beta arbutin, lucinol and its derivatives, kojic acid, resorcinol and derivatives thereof, homogentisic, methyl gentisate or homogentisate, dioic acid, D pantheteine calcium sulphonate, lipoic acid, ellagic acid, vitamin B3, linoleic acid and its derivatives, certain compounds derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include alpha and beta arbutin, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

Anti-Wrinkle Active

The cosmetic composition may include one or more anti-wrinkle actives. The term “anti-wrinkle active” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Exemplary anti-wrinkle actives may be chosen from: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof. Examples of such compounds are: adenosine and its derivatives and retinoids other than retinol (as discussed above), ascorbic acid and its derivatives such as magnesium ascorbyl phosphate and ascorbyl glucoside; nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the α-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof. In at least one case, the cosmetic composition includes adenosine derivatives, such as non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside. Other derivatives include adenosine receptor agonists such as adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

Skin Active Agent for Oily Skin

The cosmetic composition may, optionally, include a skin active agent that addresses oily skin. These agents can be sebo-regulating or antiseborrhoeic agents capable of regulating the activity of sebaceous glands. Exemplary skin active agents for addressing oily skin include: retinoic acid, retinol, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech;—extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C12-C13) sold under the name COSMACOL® ECI by Sasol; trialkyl citrate (C14-C15) sold under the name COSMACOL® ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

Preservatives

One or more preservatives may be included in the cosmetic compositions described herein. Suitable preservatives may include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), disodium EDTA, potassium sorbate, and/or grapefruit seed extract, or combinations thereof. More than one preservative may be included in the composition. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol). In one instance, the cosmetic composition has a plurality of preservatives including or chosen from disodium EDTA, phenoxyethanol, ethylhexylglycerin, tocopheryl acetate, and/or a mixture thereof.

The preservative is optionally included in an amount ranging from about 0.01 wt. % to about 5 wt. %, about 0.15% to about 1 wt. %, or about 1 wt. % to about 3 wt. %, based on the total weight of the composition.

pH Adjuster

The cosmetic composition may include one or more pH adjusters to increase or decrease the overall pH of the cosmetic composition. For example, one or more acids may be included to decrease the pH of the cosmetic composition. Examples of suitable acids for decreasing the pH of the cosmetic composition include, but are not limited to, citric acid, acetic acid, and the like. The cosmetic composition may include one or more bases, such as sodium hydroxide, potassium hydroxide and the like, to decrease the pH of the cosmetic composition. Additional or alternative acids and bases that are suitable for adjusting the pH of the cosmetic composition are readily known to one of ordinary skill in the art.

The cosmetic composition may, desirably, have a pH of pH of about 4 to about 7, preferably about 4.5 to about 6.5 or about 5.5 to about 6.5. Additionally or alternatively, the pH of the cosmetic composition may range from 4 to 7, preferably from 4.5 to 6.5, or preferably from 5.5 to 6.5. In one instance, the pH of the cosmetic composition is 6 or about 6.

The amount of the pH adjuster in the cosmetic composition may be based on the desired pH of the final cosmetic composition and/or product. For example, the total amount of the pH adjuster may range from about 0.05 to about 20 wt. %, based on the total weight of the composition. In some instances, the total amount of pH adjuster is from about 0.05 to about 15 wt. %, about 0.5 to about 10 wt. %, about 1 to about 5 wt. %, about 1.5 to about 4 wt. %, or about 2.0 to about 3 wt. %, including ranges and sub-ranges there between, based on the total weight of the composition. Additionally or alternatively, the cosmetic compositions may include an amount of pH adjuster ranging from 0.05 to 15 wt. %, 0.5 to 10 wt. %, 1 to 5 wt. %, 1.5 to 4 wt. %, or 2.0 to 3 wt. %, including ranges and sub-ranges there between, based on the total weight of the composition.

Water

The total amount of water in the cosmetic composition can vary, but is typically about 30 to about 95 wt. %, based on the total weight of the cosmetic composition. In some instances, total amount of water is about 30 to about 90 wt. %, about 30 to about 85 wt. %, about 30 to about 80 wt. %, about 35 to about 90 wt. %, about 35 to about 85 wt. %, about 35 to about 80 wt. %, about 40 to about 90 wt. %, about 40 to about 85 wt. %, about 40 to about 80 wt. %, about 45 to about 90 wt. %, about 45 to about 85 wt. %, about 45 to about 80 wt. %, about 50 to about 90 wt. %, about 50 to about 85 wt. %, about 50 to about 80 wt. %, about 55 to about 90 wt. %, about 55 to about 85 wt. %, about 55 to about 80 wt. %, about 60 to about 90 wt. %, about 60 to about 85 wt. %, about 60 to about 80 wt. %, about 65 to about 90 wt. %, about 65 to about 85 wt. %, or about 65 to about 80 wt. %, based on the total weight of the cosmetic composition.

The cosmetic compositions of the instant disclosure are preferably stable. The term “stable” as used herein means that the cosmetic composition does not visually phase separate or crystallize and does not completely degrade

The instant disclosure also relates to methods of using the cosmetic compositions described herein. For example, the cosmetic compositions can be used in a method that comprises applying the cosmetic compositions to this skin of humans. In some cases, the composition is applied to the face. Furthermore, the cosmetic composition can be used in methods for treating dryness of the skin, repairing damage to skin (for example, damage from photoaging), and for diminishing the appearance of wrinkles, dark spots, and uneven skin texture of skin. The aforementioned methods are non-therapeutic.

The cosmetic composition may be applied once per day, twice per day, or more than once or twice per day. In some cases, the composition is applied in the evenings before bed. In other cases, the compositions are applied in the morning. In still other cases, the composition may be applied immediately after washing the skin. The compositions may be used once, or for a series of days, weeks, or months. For example, the compositions may be used daily for a period of 1, 2, 3, 4, 5, 6, 7, 8 or more weeks, or months.

### EXAMPLES

Implementation of the present disclosure is provided by way of the following examples. The following examples serve to elucidate aspects of the technology without being limiting in nature.

### Example 1

### Inventive Compositions

TABLE 1

| | | Inventive | Inventive |
|---|---|---|---|
| Phase | INCI US | Ex. 1* | Ex. 2* |
| Phase A1 | WATER/AQUA | Q.S. | Q.S. |
| | POLYACRYLATE | 0.80 | 0.80 |
| | CROSSPOLYMER-6 | | |
| | SCLEROTIUM GUM | 0.30 | 0.30 |
| | SODIUM HYALURONATE | 0.10 | 0.10 |
| Phase A2 | INULIN LAURYL CARBAMATE | 1 | 1 |
| | TRISODIUM | 0.11 | 0.11 |
| | ETHYLENEDIAMINE | | |
| | DISUCCINATE | | |
| | PHENOXYETHANOL | 0.60 | 0.60 |
| | CAPRYLYL GLYCOL | 0.30 | |
| | CHLORPHENESIN | | 0.20 |
| | BUTYLENE GLYCOL | 3 | 4 |
| | PENTYLENE GLYCOL | 3 | |
| | HEXYLENE GLYCOL | | 1 |
| Phase B | ISONONYL ISONONANOATE | 3 | 3 |
| | DICAPRYLYL CARBONATE | 1 | 0.80 |
| | GLYCINE SOJA (SOYBEAN) | 0.60 | 0.80 |
| | OIL/GLYCINE SOJA OIL | | |
| | OCTYLDODECANOL | 2.40 | 2.50 |
| | ISOPROPYL LAUROYL | 1.50 | 1.50 |
| | SARCOSINATE | | |
| | ISOCETYL STEAROYL | 0.40 | 0.30 |
| | STEARATE | | |
| | HYDROGENATED LECITHIN | 0.60 | 0.60 |
| | C14-22 ALCOHOLS (and) C12- | 1.20 | 1.20 |
| | 20 ALKYL GLUCOSIDE | | |
| | PENTAERYTHRITYL TETRA-DI- | 0.20 | 0.20 |
| | T-BUTYL | | |
| | HYDROXYHYDROCINNAMATE | | |
| | PHENYLETHYL RESORCINOL | 0.30 | |
| | HYDROXYACETOPHENONE | 0.50 | 0.50 |
| | CAPRYLOYL SALICYLIC ACID | 0.30 | 0.30 |
| Phase C | TOCOPHEROL | 0.50 | 0.50 |
| Phase D | ASCORBIC ACID | 10 | 10 |
| | WATER/AQUA | 3 | 3 |
| | SODIUM HYDROXIDE | 2.20 | 2.20 |
| Phase E | ACRYLAMIDE/SODIUM | 1 | 1 |
| | ACRYLOYLDIMETHYLTAURATE | | |
| | COPOLYMER (and) | | |
| | ISOHEXADECANE (and) | | |
| | POLYSORBATE 80 | | |
| Phase F | METHYL METHACRYLATE | 0.90 | 0.90 |
| | CROSSPOLYMER | | |
| | BORON NITRIDE | 0.60 | 0.60 |

*% of Active ingredients

The Inventive Examples were prepared according to the procedure as follows:

Polymers in phase A1 were dispersed in water with agitation until a clear gel with no powder or lumps were formed. Then, components of phase A2 were added to the resultant gel with agitation with yield aqueous phase A. Phase A was then heated to about 75 C. Separately, phases B and C were combined and heated to 75 C until all materials melted, dispersed, and/or solubilized. Phases B and C were then added slowly to phase A with sitting and homogenization that continued until a fine emulsion formed. The resultant formula was then cooled down. Upon reaching <30 C, the components of phase D were added, followed by the components of phase E, with homogenization to combine. Finally, phase F was added and formula was homogenized. Process was complete when a fine emulsion with well-dispersed powder resulted, and batch reached about 25 C.

### Example 2

### Evaluation of Vitamin C Chemical Stability

Inventive Examples 1 and 2 were formulated for purposed of containing high levels of actives, mainly high levels of pure Vitamin C. In order to evaluate the stability of the pure Vitamin C in the formulation, various antioxidants and chelators were tested in these emulsions (i.e. Comparative Examples). The inventive et comparative examples are presented in Table 2.

TABLE 2
Inventive and Comparative Examples

| | Control | Inv. | Inv. | Comp. | Comp. | Comp. | Comp. | Comp. | Comp. | Comp. | Comp. |
|---|---|---|---|---|---|---|---|---|---|---|---|
| INCI US | Ex.* | Ex1* | Ex. 2* | Ex. 1* | Ex. 2* | Ex. 3* | Ex. 4* | Ex. 5* | Ex. 6* | Ex. 7* | Ex. 8* |
| ASCORBIC ACID | 10.00 | 10.00 | 10.00 | 10.00 | 10.00 | 10.00 | 10.00 | 10.00 | 10.00 | 10.00 | 10.00 |
| TOCOPHEROL | 0.50 | 0.50 | 0.50 | 0.50 | | 0.50 | 0.50 | 0.50 | 0.50 | 0.50 | 0.50 |
| HYDROXYACETOPHENONE | 0.50 | 0.50 | 0.50 | | 0.50 | 0.50 | 0.50 | 0.50 | 0.50 | 0.50 | 0.50 |
| PENTAERYTHRITYL | | 0.20 | 0.20 | 0.20 | 0.20 | | | | | | |
| TETRA-DI-T-BUTYL | | | | | | | | | | | |
| HYDROXYHYDROCINNAMATE | | | | | | | | | | | |
| FERULIC ACID | | | | | | 0.50 | | | | | |
| DIMETHYL SULFONE | | | | | | | 1.00 | | | | |
| DIETHYLHEXYL | | | | | | | | 0.50 | | | |
| SYRINGYLIDENEMALONATE | | | | | | | | | | | |
| TRISODIUM | 0.11 | 0.11 | 0.11 | | 0.11 | 0.11 | 0.11 | 0.11 | 0.11 | 0.11 | 0.11 |
| ETHYLENEDIAMINE | | | | | | | | | | | |
| DISUCCINATE | | | | | | | | | | | |
| SODIUM PHYTATE | | | | | | | | | 0.50 | | |
| TETRASODIUM GLUTAMATE | | | | | | | | | | 0.25 | |
| DIACETATE | | | | | | | | | | | |
| CHLOROGENIC ACIDS | | | | | | | | | | | 0.50 |

*% of Active ingredients

After formulation, each example formula was filled into an aluminum tube and chemical stability was observed. The analytical team metered the amount of vitamin C and it was measured over time by an analytical method. The results are presented in the table below.

TABLE 3
Vitamin C Chemical Stability

| | | Vitamin C chemical stability | | | | | | |
|---|---|---|---|---|---|---|---|---|
| | | | T1M | T1M | T2M | T2M | % | |
| Examples | Description | Initial | 25 | 45 | 25 | 45 | Remaining | Comments |
| Control | Base formula | >10.90% | 10.90% | 10.10% | 9.71% | 8.44% | 77.43% | |
| Ex. | | | | | | | | |
| Comp. | 0.5% ferulic acid | >10.30% | 10.30% | 9.50% | 9.80% | 8.10% | 78.64% | |
| Ex. 3 | (AOX) | | | | | | | |
| Comp. | 1% Dimethyl | 11.30% | 9.93% | 9.37% | 9.84% | 8.82% | 78.05% | |
| Ex. 4 | sulfone (AOX) | | | | | | | |
| Comp. | 0.5% Oxynex ST | 11.16% | 10.15% | 9.43% | 9.66% | 9.00% | 80.65% | |
| Ex. 5 | (AOX) | | | | | | | |
| Inv. | 0.2% Tinogard TT | 10.94% | 10.22% | 9.50% | 9.95% | 9.50% | 86.84% | Best stability |
| Ex. 1 | (AOX) | | | | | | | |
| Inv. | 0.2% Tinogard TT | 9.54% | 9.00% | 8.49% | 9.73% | 9.20% | 96.44% | Best stability |
| Ex. 2 | (AOX) | | | | | | | confirmation; similar |
| | | | | | | | | to Inv. Ex. 1 |
| Comp. | 0.5% sodium | 10.53% | 9.82% | 9.16% | 10% | 8.97% | 85.19% | |
| Ex. 6 | phytate (Chelator) | | | | | | | |
| Comp. | 0.25% Dissolvine | 10.54% | 9.82% | 8.97% | 10.11% | 8.62% | 81.78% | |
| Ex. 7 | (Chelator) | | | | | | | |
| Comp. | 0.5% chlorogenic | 11.01% | 9.91% | Tube | 10.36% | Tube | Tube | |
| Ex. 8 | acid (Chelator) | | | burst | | burst | burst | |

Table 3 above showed the evaluation of chemical stability of various antioxidants (AOX) and chelators in the representative formulation. Finished formulations in sealed aluminum tubes were placed in controlled chambers at 25 C and 45 C. A freshly made tube was also submitted to analysis to obtain the starting baseline active concentration (i.e., T0). At 1 month (T1M) and 2 months (T2M) in each temperature controlled chamber, a tube of formula was submitted for quantitative active analysis. Initial (TOM) was then compared with aged sample at elevated temperature (T2M45C).

In the case of the Control Example that only contained a chelator (i.e., EDDS=Trisodium Ethylenediamine Disuccinate), it was observed that the Vitamin C was not very stable and that after T2M at 45° C., the percentage of Vitamin C remaining was about 77.43%. Then, different Antioxidants as well as different chelators were evaluated and demonstrated that all the Antioxidants and chelators were not equal. Inventive Ex. 1 and Inventive Ex. 2 exhibited the best stability for the Vitamin C, proving that the presence of Pentaerythrityl Tetra-Di-T-Butyl Hydroxyhydrocinnamate (i.e., Tinogard TT) associated with a chelator exhibited an improvement of Vit C stability. Additionally, when Pentaerythrityl Tetra-Di-T-Butyl Hydroxyhydrocinnamate (i.e., Tinogard TT) was kept in the formula, but hydroxyacetophenone and a chelator were removed, stability was poor (See Comp. Ex. 1 and Comp. Ex. 2). Thus, the combination of Pentaerythrityl Tetra-Di-T-Butyl Hydroxyhydrocinnamate, hydroxyacetophenone, and a chelator were required to obtain high chemical stability.

In the Inventive Examples, pure Vitamin C was able to be stabilized with high chemical stability (>85%) even after 2 months at 45 C. This was in contrast to other formulas that didn't contain the combination of Pentaerythrityl Tetra-Di-T-Butyl Hydroxyhydrocinnamate, Hydroxyacetophenone and a chelating agent.

TABLE 4
Vitamin C Chemical Stability of Comparative Examples

| | | Vitamin C chemical stability | | | | | | |
|---|---|---|---|---|---|---|---|---|
| | | | T1M | T1M | T2M | T2M | % | |
| Formulas | Description | Initial | 25 | 45 | 25 | 45 | Remaining | Comments |
| Comp | 0.2% Tinogard TT | 9.36% | 7.55% | 7.73% | 5.56% | 5.92% | 63.25% | Poor stability; shows that |
| Ex. 1 | without | | | | | | | hydroxyacetophenone |
| | hydroxyacetophenone | | | | | | | and a chelator (e.g., |
| | or EDDS | | | | | | | EDDS) is needed |
| Comp. | 0.2% Tinogard TT | 10.11% | 11.43% | 10.79% | 11.41% | 10.40% | 102.87% | Best stability; shows |
| Ex. 2 | without Vitamin E | | | | | | | Vit E is not needed |

In Table 4, Comparative Ex. 1 demonstrated that the combination of Pentaerythrityl Tetra-Di-T-Butyl Hydroxyhydrocinnamate, hydroxyacetophenone, and a chelator were required to obtain high chemical stability. It was also demonstrated with Comparative Ex. 2 that the presence of Vitamin E is not necessary in order to stabilize the high level of Vitamin C.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the cosmetic compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

The term “treat” (and its grammatical variations) as used herein refers to the application of the compositions of the present disclosure onto the surface of keratinous substrates such as skin, in particular, the skin of the head, face, and neck.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of alkoxylated compounds, for example, ethoxylated thickeners and/or ethoxylated surfactants. Likewise, a particular composition may be free or essentially free of sulfates, such as sulfate surfactants.

While the instant disclosure has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the instant disclosure without departing from the essential scope thereof. Therefore, it is intended that the instant disclosure is not limited to the particular embodiment disclosed as the best mode contemplated for carrying out this instant disclosure, but that the instant disclosure will include all embodiments falling within the scope of the appended claims.

1. A cosmetic composition comprising:
(a) from about 2 to about 20 wt. % of ascorbic acid;
(b) di-t-butyl pentaerythrityl tetrahydroxycinnamate;
(c) hydroxyacetophenone; and
(d) a chelating agent; and
wherein the cosmetic composition is an oil-in-water emulsion, and all weight percentages are based on the total weight of the cosmetic composition.

2. The cosmetic composition of claim 1, wherein the cosmetic composition is in the form of an oil-in-water emulsion.

3. The cosmetic composition of claim 1, wherein di-t-butyl pentaerythrityl tetrahydroxycinnamate is present from about 0.05 to about 0.5 wt. % based on the total weight of the cosmetic composition.

4. The cosmetic composition according to claim 1, wherein the hydroxyacetophenone is present from about 0.1 to about 3 wt. % based on the total weight of the cosmetic composition.

5. The cosmetic composition according to claim 1, wherein the chelating agent is chosen from Trisodium Ethylenediamine Disuccinate, Sodium Phytate, Tetrasodium Glutamate Diacetate, and mixture thereof.

6. The cosmetic composition according to claim 1, the chelating agent is Trisodium Ethylenediamine Disuccinate.

7. The cosmetic composition according to claim 1, wherein the chelating agent is present from about 0.01 to about 1 wt. % based on the total weight of the cosmetic composition.

8. The cosmetic composition according to claim 1, further comprising one or more fatty compounds.

9. The cosmetic composition of claim 1, wherein the one or more fatty compounds is chosen from fatty alcohols, fatty acids, fatty esters, oils, waxes, derivatives thereof, and mixtures thereof.

10. The cosmetic composition of claim 9 comprising one or more fatty ester chosen from ethoxylated fatty esters, sorbitan fatty esters, esters of stearates, esters of behenates, esters of arachidates, esters of palmitates, fatty acid esters of a sugar, and mixtures thereof.

11. The cosmetic composition of claim 10 comprising one or more fatty ester chosen from purcellin oil (cetearyl octanoate), isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, and pentaerythritol esters, and mixtures thereof.

12. The cosmetic composition of claim 6, wherein the one or more fatty compounds comprises isononyl isononanoate, caprylic/capric triglyceride, Glycine soja (soybean) oil or mixtures thereof.

13. The cosmetic composition of claim 1 comprising about 0.5 to about 20 wt. % of fatty phase.

14. The cosmetic composition of claim 1, further comprising one or more polyols.

15. The cosmetic composition of claim 14, wherein the one or more polyols is chosen from caprylyl glycol, pentylene glycol, butylene glycol, hexylene glycol, glycerin, dipropylene glycol, propylene glycol, and mixtures thereof.

16. A cosmetic composition comprising:
(a) from about 2 to about 20 wt. % of ascorbic acid;
(b) from about 0.05 to about 0.5 wt. % of di-t-butyl pentaerythrityl tetrahydroxycinnamate;
(c) from about 0.01 to about 3 wt. % of hydroxyacetophenone; and
(d) from about 0.01 to about 2 wt. % of a chelating agent chosen from Trisodium Ethylenediamine Disuccinate, Sodium Phytate, Tetrasodium Glutamate Diacetate; and
wherein the cosmetic composition is an oil-in-water emulsion, and all weight percentages are based on the total weight of the cosmetic composition.

17. A method for treating skin comprising applying a cosmetic composition of claim 1 to the skin.
